(19) United States
(12) Patent Application Publication
(10) Pub. No.: US 2026/0351339 A1
(43) Pub. Date: Nov. 5, 2026

(54) SYSTEMS AND MTEHODS FOR VALIDATION OF PROTEOMIC MODELS

(71) Applicant: SOMALOGIC OPERATING CO., INC., Boulder, CO (US)

(72) Inventors: Yolanda HAGAR, Boulder, CO (US); Natasha KURESHI, Boulder, CO (US); Leigh ALEXANDER, Boulder, CO (US); Mike HINTERBERG, Boulder, CO (US); Jeremy PRIMUS, Boulder, CO (US); Gargi DATTA, Boulder, CO (US); Amy ZHANG, Boulder, CO (US); Caleb SCHEIDEL, Boulder, CO (US)

(21) Appl. No.: 19/119,463

(22) PCT Filed: Oct. 11, 2023
(86) PCT No.: PCT/US2023/076612

§ 371 Date: Apr. 9, 2025

(51) Int. Cl. G16B 20/00; G06F 30/27; G16B 40/20

(52) U.S. Cl. CPC G16B 20/00; G06F 30/27; G16B 40/20

(57) ABSTRACT

Methods, systems, and computer-readable media for developing or validating resilient proteomics models can estimate an impact of different contexts on the input data to the model, then develop or validate the proteomics models using the estimated impact. An exemplary method for validating a proteomics model can estimate protein level noise using a control dataset and a treatment dataset. The control dataset and treatment dataset can be different from a training dataset used to generate the model. A validation dataset can be generated using the training dataset and the estimated protein level noise. The validation dataset can include adjusted protein level measurements. A set of predictions can be generated by applying the validation dataset to a proteomics model trained using the training dataset. The set of predictions can be used to determine the validity of the proteomics model.

### CROSS-REFERENCE TO RELATED APPLICATIONS

This application claims the benefit of U.S. Provisional Application No. 63/415,978, filed Oct. 13, 2022, which is incorporated herein by reference in its entirety.

### BACKGROUND

Aptamer-based assays can simultaneously measure protein levels for thousands of proteins in samples drawn from patients. However, due in part to the nature of such assays, measured protein levels can vary with changes in sample characteristics, changes in sample handling, or changes in assay protocol. These changes may affect the outcome of proteomics models that use the measured protein levels as input features. Therefore, changes to sample characteristics, sample handling, and assay protocol can result in a decreased performance of such proteomics models.

Changes in sample characteristics, sample handling, or assay protocol may therefore necessitate re-validation of a corresponding proteomics model. However, re-validation of this proteomics model may require samples representative of a full range of anticipated inputs. Such samples may no longer be available or may be difficult to obtain. Difficulties in obtaining such samples may also inhibit the development of proteomics models resilient to changes in sample characteristics, sample handling, or assay protocol.

### SUMMARY

Certain embodiments of the present disclosure relate to development of proteomics models resilient to changes in input data, such as sample characteristics, sample handling, or assay protocol. Consistent with disclosed embodiments, the effects of such changes on the input data can be estimated. The estimated effects can then be used to generate updated data for validating or training a proteomics model.

The disclosed embodiments include a system for validating proteomics models. The system can include at least one processor and at least one non-transitory computer-readable medium. The computer-readable medium can contain instructions that, when executed by the at least one processor, cause the system to perform operations. The operations can include obtaining a control dataset including protein level measurements. The operations can further include obtaining a treatment dataset including protein level measurements. The operations can further include obtaining a training dataset including protein level measurements. The operations can further include estimating protein level noise using the control dataset and the treatment dataset. The operations can further include generating a validation dataset using the training dataset and the estimated protein level noise. The validation dataset can include adjusted protein level measurements. The operations can further include generating a set of predictions by applying the validation dataset to a proteomics model trained using the training dataset. The operations can further include determining a validity indication using the set of predictions and providing the validity indication.

The disclosed embodiments include a system for developing proteomics models. The system can include at least one processor and at least one non-transitory computer-readable medium. The computer-readable medium can contain instructions that, when executed by the at least one processor, cause the system to perform operations. The operations can include obtaining a proteomics model trained to generate a prediction based on training protein level measurements generated from training aliquots obtained in a first context. The operations can further include generating treatment protein level measurements from treatment aliquots or samples obtained in second contexts. The operations can further include determining a dependence of the proteomics model on differences between the second contexts. The operations can further include updating the proteomics model based on the determined dependence.

The disclosed embodiments further include corresponding methods for validating and/or developing proteomics models; and non-transitory, computer-readable media containing executable instructions for performing such methods.

The foregoing general description and the following detailed description are exemplary and explanatory only and are not restrictive of the claims. Other systems, methods, and computer-readable media are also discussed within.

### BRIEF DESCRIPTION OF THE DRAWINGS

The accompanying drawings, which are incorporated in and constitute a part of this specification, illustrate several embodiments and, together with the description, serve to explain the disclosed principles. In the drawings:

FIG. 1 depicts an exemplary pipeline for developing, validating, and deploying proteomics models for predicting health information using aptamer-based blood tests, consistent with disclosed embodiments.

FIGS. 2A to 2H provide a high-level depiction of stages in an exemplary aptamer-based serum or plasma assay, consistent with disclosed embodiments.

FIGS. 3A and 3B depict an exemplary challenge arising in the validation of proteomics models for predicting health information using aptamer-based serum or plasma tests, consistent with disclosed embodiments.

FIGS. 4A to 4F depict exemplary model-resilience challenges arising in the training of proteomics models for predicting health information using aptamer-based blood tests, consistent with disclosed embodiments.

FIG. 5 depicts an exemplary process for validating proteomics models, consistent with disclosed embodiments.

FIG. 6 depicts an exemplary process for developing proteomics models, consistent with disclosed embodiments.

FIGS. 7A to 7D depict the effect of differences in sample handling on the output of a binary endpoint proteomics model, consistent with disclosed embodiments.

FIGS. 8A to 8D depict the effect of differences in sample handling on the output of a continuous endpoint proteomics model, consistent with disclosed embodiments.

FIGS. 9A to 9D depict the effect of differences in sample handling on the predictive performance of predictions made by a proteomics model, consistent with disclosed embodiments.

### DETAILED DESCRIPTION

In the following detailed description, numerous specific details are set forth in order to provide a thorough understanding of the disclosed example embodiments. However, it will be understood by those skilled in the art that the principles of the example embodiments may be practiced without every specific detail. Well-known methods, procedures, and components have not been described in detail so as not to obscure the principles of the example embodiments. Unless explicitly stated, the example methods and processes described herein are neither constrained to a particular order or sequence nor constrained to a particular system configuration. Additionally, some of the described embodiments or elements thereof can occur or be performed simultaneously, at the same point in time, or concurrently. Reference will now be made in detail to the disclosed embodiments, examples of which are illustrated in the accompanying drawings.

Proteomics, as used herein, can concern or involve the quantitative assessment of proteins present in a sample obtained from a human or non-human animal. Proteomics can concern classifications or predictions drawn from large-scale (e.g., concerning hundreds to thousands) measurement of protein levels (e.g., presence, amount, concentration, or the like). In this manner, proteomics can be similar to genomics, but applied to the realm of proteins.

A predictive model, as used herein, can be model that maps a set of inputs to an output value or category. In some embodiments, the predictive models can be machine-learning models, which can be trained using training data to generate appropriate outputs. The predictive models can be trained using supervised methods, semi-supervised methods, unsupervised methods, or reinforcement learning methods. In some embodiments, the predictive models can be statistical models that estimate a relationship, using training data, between independent variables (e.g., protein levels in a biological sample) and dependent variables (e.g., health information values). Exemplary predictive models include regression models (e.g., penalized regression models), support vector machines, decision trees or forests, linear discriminant analysis, clustering models, nearest neighbor models, neural network models, ensemble models including one or more of any of the foregoing models, or the like.

A proteomics model, as used herein, can be a predictive model configured to receive protein level measurements and output classifications or predictions based at least in part on those measurements.

Health information can include a probability of a health outcome (e.g., cardiovascular disorder, dementia, kidney disease, or the like) occurring within a particular time frame (e.g., within six months, a year, four years, a decade, two decades, or longer) an indication of present health status (e.g., percentage body fat, a basal metabolic rate, lean muscle mass, aerobic fitness, visceral fat content, glomerular filtration rate, presence or absence of excess liver fat, glucose tolerance, or the like), a behavioral health prediction (e.g., predicted weekly alcohol consumption, or the like), or the like.

Consistent with disclosed embodiments, providing models, data, or instructions can include direct (e.g., between source and target) or indirect (e.g., through intermediaries) provision of such models, data, or instructions. Providing a model, data, or instructions can include providing by value the model, data, or instructions or providing by reference to a location storing the model, data, or instructions.

Consistent with disclosed embodiments, performance measures can be used to quantify the performance of a predictive model. In some embodiments, performance measures can quantify the agreement between two predictions (e.g., to evaluate reproducibility or the effect of differences in input data on the predicted output). Such performance measures can include Lin's concordance correlation coefficient, Pearson's correlation coefficient, or other suitable correlation measures. In some embodiments, performance measures can quantify the agreement between predictions and a ground truth. In some embodiments, such performance measures can quantify this agreement in terms of a relationship between true positive rate and false positive rate, as parameters of the predictive model are varied (e.g., a discrimination threshold, or the like). Such measures can include a receiver operating characteristic (ROC) curve, or derivative performance measures, such as area under the ROC curve (AUC). In some embodiments, such performance measures can include or depend upon numbers of true positives, true negative, false positives, or false negatives for a classification task. In some embodiments, such measures can include sensitivity, specificity, precision, false negative rate, false positive rage, prevalence, accuracy, F1 score, or other such measures. In some embodiments, such performance measures can include or depend upon a per-class comparison between predictions and a ground truth, such as a confusion matrix or the like.

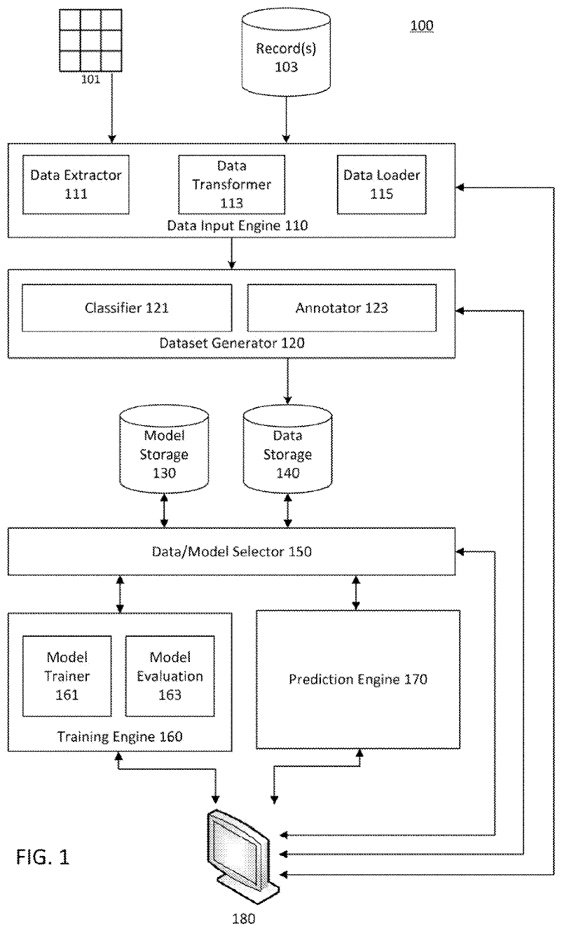

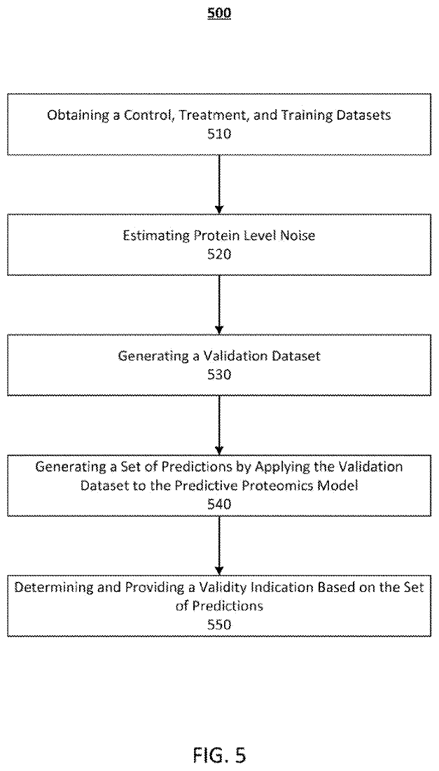

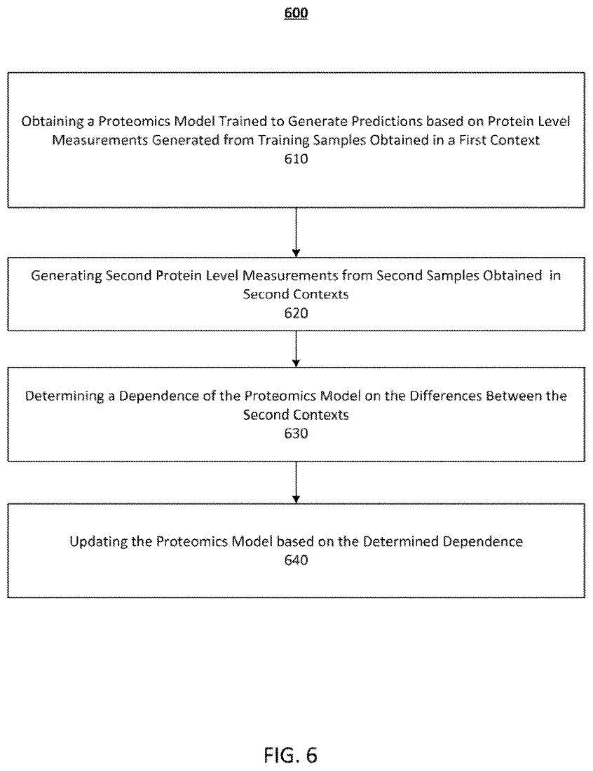

FIG. 1 depicts an exemplary pipeline 100 for developing, validating, and deploying proteomics models for predicting health information using aptamer-based blood tests, consistent with disclosed embodiments. Pipeline 100 can include components from which data is originally obtained, such as measurement system 101 or records 103. Pipeline 100 can include components, such as data input engine 110 or dataset generator 120, for the collection and preparation of the obtained data. Pipeline 100 can include components, such as data storage 140 and model storage 130, for the storage of prepared data and machine learning models. Pipeline 100 can include training engine 160 for generating trained machine learning models using obtained data and models. Pipeline 100 can include prediction engine 170 for using trained machine learning models to predict health information. Components of pipeline 100 can be managed and configured through a user device 180. User device 180 can also be used to display outputs of other components, original or prepared data, trained or untrained models, or predicted health information. A user can interact with components of pipeline 100 to perform model validation and development, consistent with disclosed embodiments. In some embodiments, a user can interact with training engine 160 (or another suitable component of pipeline 100) to perform model validation as described with regards to FIG. 5 or model development as described with regards to FIG. 6. Overall, pipeline 100 can provide a convenient, scalable, platform for developing, validating, and deploying proteomics models, as disclosed herein.

Measurement system 101 can be a device suitable for obtaining data indicating protein presence or concentration in a biological sample. For convenience of discussion, measurement system 101 is described herein as a microarray scanner. Such a scanner can be configured to measure fluorescence at predetermined locations corresponding to different proteins on a microarray. The intensity of the fluorescence can indicate a concentration of the protein in a biological sample used to prepare the microarray. In some instances, multiple locations can correspond to the same protein. Fluorescence intensity data for these multiple locations can be combined to better estimate the concentration of the protein in the sample. As may be appreciated, pipeline 100 is not limited to embodiments in which measurement system 101 is a microarray scanner. Furthermore, in some embodiments, rather than providing data directly to data input engine 110, measurement system 101 can provide data to record(s) 103. Data input engine 110 can then obtain this data from record(s) 103.

Consistent with disclosed embodiments, record(s) 103 can include one or more storage locations for data usable by pipeline 100 to predict healthcare outcomes. In some embodiments, such data can include fluorescence intensity data generated by measurement system 101 from biological samples. In various embodiments such data can include medical record information from the patients from which the biological samples were obtained. Such medical record information can include medical records, case notes, request or requisition information (e.g., pertaining to the sample or to the prediction to be performed by pipeline 100) provided by a physician or other clinician. In some embodiments, the medical record information can include class or data label information corresponding to the samples (e.g., for use in generating training datasets).

Consistent with disclosed embodiments, the medical record information can be associated with health information. For example, in a cancer screening or diagnosis setting, the medical record information can include data associated with a cancer type, a cancer prevalence, a cancer prognosis, or a cancer stage.

Consistent with disclosed embodiments, the medical record information can include information suitable for use in generating personalized predictive models. For example, the medical record information can indicate patient demographic or health characteristics, such as age, gender, race/ethnicity, height/weight of the patient. As an additional example, the medical record information can indicate patient medical history, such as a history of medical treatment, clinical visits, or surgical history. As an additional example, the medical record information can indicate behavioral factors that could affect patient health, such as smoking history, alcohol consumption, drug use, diet type, etc. As an additional example, the medical record information can include data obtained from a biological sampling from the patient, such as data relating to a sample of blood, plasma, serum, or urine. As an additional example, the medical record information can indicate family history data, genetic data, or immunological data.

Consistent with disclosed embodiments, data input engine 110 can be configured to retrieve data from a variety of data sources (e.g., measurement system 101, record(s) 103, or other suitable sources) and process the data for use by other components of pipeline 100. In some embodiments, data input engine 110 can include data extractor 111, data transformer 113, and data loader 115.

Consistent with disclosed embodiments, data extractor 111 can receive or retrieve data from measurement system 101, record(s) 103, or other suitable sources. As described herein, measurement system 101 can be a diagnostic system configured to obtain fluorescence values corresponding to protein presence or concentrations in a biological sample. Similarly, record(s) 103 can be one or more databases or data storage locations containing data concerning the biological samples used to generate the fluorescence values. The disclosed embodiments are not limited to any particular format of the obtained data, or method for obtaining this data. For example, the obtained data can be or include structured data or unstructured data. Data extractor 111 can interact with the various data sources, receive or retrieve the relevant data, and provide that data to data transformer 113.

Consistent with disclosed embodiments, data transformer 113 can receive data from data extractor 111 and process the data into standard formats. In some embodiments, data transformer 113 can normalize data such as dates or numerical values based on specific units of measure. For example, measurement system 101 can store dates in day-month-year format, while records 103 can include records that store dates in year-month-day format, or various records that store measurement in differing units (e.g., body weights in kilograms or pounds, fluorescence in absolute magnitude or log10 magnitude, or the like). In this example, data transformer 113 can modify the data provided through data extractor 111 into a consistent date format or standardized unit format, respectively. Accordingly, data transformer 113 can effectively clean the data provided through data extractor 111 so that all of the data, although originating from a variety of sources, has a consistent format.

Moreover, data transformer 113 can extract additional data points from the data. For example, data transformer 113 can process a date in year-month-day format by extracting separate data fields for the year, the month, and the day. Data transformer 113 can also perform other linear and non-linear transformations (e.g. logarithmic transformations of continuous data, or the like) and extractions on categorical and numerical data, such as normalization and centering the data about the mean of the data. Data transformer 113 can provide the transformed or extracted data to data loader 115.

Consistent with disclosed embodiments, data loader 115 can receive the normalized data from data transformer 113. Data loader 115 can merge the data into varying formats depending on the specific requirements of dataset generator 120. Data loader 115 can then provide the processed data to dataset generator 120 (or to a suitable data storage, from which dataset generator 120 can retrieve the data).

Consistent with disclosed embodiments, dataset generator 120 can be configured to generate datasets from data processed by data input engine 110. In some embodiments, training engine 160 or prediction engine 170 can be configured to expect datasets having a particular structure. Dataset generator 120 can be configured to format data into that particular structure. For example, dataset generator 120 can be configured to collect observations, associate the observations with corresponding training labels or metadata, and store the observations and metadata in data storage 140.

In some embodiments, dataset generator 120 can be configured to extract features from data received from data input engine 110. A feature can be a property or characteristics of a phenomenon. For example, the presence or absences of a fluorescent value in excess of a threshold at a location corresponding to a particular protein can be a feature. As an additional example, the average fluorescence value over samples on a biochip can be a feature. Features can be determined based on the domain, data type of a category, or many other factors associated with data stored in a data structure. Additionally, a feature can represent information about multiple data records in a data set or information about a single category in a data record. Moreover, multiple features can be produced to represent the same data.

In some embodiments, dataset generator 120 can include a classifier 121 and an annotator 123. Consistent with disclosed embodiments, classifier 121 can be configured to create classes from data provided by dataset generator 120. In some embodiments, such classes can be data labels used for training a predictive model. For example, a set of fluorescence data indicating protein levels can be associated with a medical record of a patient. In this example, the classifier can extract health information from the medical record. For example, the classifier can determine that a patient experienced a cardiovascular event within a particular amount of time following acquisition of a blood sample used to generate the fluorescence data. As an additional example, the classifier can identify a glomerular filtration rate of the patient from the medical record. In some embodiments, the classifier can be or include a natural language processing engine.

Consistent with disclosed embodiments, classifier 121 can be configured to accept classifications provided by a user through user device 180. For example, classifier 121 can be configured to provide data (or metadata concerning the data) received from data input engine 110 to user device 180 for display. In response, classifier 121 can receive class information. For example, classifier 121 can provide medical records associated with a set of fluorescence data for display to user device 180. In response, a user can interact with user device 180 to provide indications of health information based on the provided medical records. As may be appreciated, such functionality is not limited to medical records, but could be another information used to generate training labels for the data.

Consistent with disclosed embodiments, annotator 123 can be configured to receive data and associated value or class information from dataset generator. Annotator 123 can be configured to create a suitably formatted entry that associates the value or class information with the data. For example, given an array or matrix of fluorescence values and a glomerular filtration rate, annotator 123 can create an object including a “response_value” key and an “protein_data_input” key. The glomerular filtration rate can be stored with the “response_value” key and the array or matrix of fluorescence values “protein_data_input” key. In this example, training engine 160 or prediction engine 170 can expect, or be configured to expect, observations having such a format. As an additional example, given sets of fluorescence values obtained from blood samples drawn from patients and dates the patients suffered heart attacks, annotator 123 can create a relational database with rows corresponding to patients, one column storing whether the patient suffered a heart attack, another column storing when the patient suffered the heart attack, and the remaining columns storing the fluorescence values.

Consistent with disclosed embodiments, model storage 130 can be a storage location for predictive models usable by training engine 160 or prediction engine 170. The disclosed embodiments are not limited to any particular implementation of data storage 140. Consistent with disclosed embodiments, data storage 140 can be implemented using one or more relational databases, object-oriented or document-oriented databases, tabular data stores, graph databases, distributed file systems, or other suitable data storage options.

Consistent with disclosed embodiments, data storage 140 can be a storage location for prepared datasets usable by training engine 160 or prediction engine 170. The disclosed embodiments are not limited to any particular implementation of data storage 140. Consistent with disclosed embodiments, data storage 140 can be implemented using one or more relational databases, object-oriented or document-oriented databases, tabular data stores, graph databases, distributed file systems, or other suitable data storage options.

Consistent with disclosed embodiments, data/model selector 150 can be configured to access model storage 130 or data storage 140 to retrieve predictive models or datasets, respectively. In some embodiments, data/model selector 150 can provide an abstraction layer for training engine 160 or prediction engine 170. In some embodiments, data/model selector 150 can be configured to control access to model storage 130 or data storage 140.

Consistent with disclosed embodiments, training engine 160 can be configured to train, or create and train, predictive models. Training engine 160 can be configured to obtain existing models from model storage 130 or training datasets from data storage 140. In some embodiments, training engine 160 can be configured to interact with data/model selector 150 to obtain the existing models or training datasets. Training engine 160 can be configured to store trained predictive models in model storage 130. In some embodiments, training engine 160 can be configured to interact with data/model selector 150 to store the trained predictive models in model storage 130.

Consistent with disclosed embodiments, training engine 160 can include model trainer 161 and model evaluation 163. Training engine 160 can be configured to train a predictive model using model trainer 161 and then determine performance metric values for the predictive model using model evaluation 163. In some embodiments, training engine 160 can automatically update the predictive model being trained based on the performance metric values. In various embodiments, training engine 160 can update the predictive model being trained in response to user input provided through user device 180. Updating the predictive model can include one or more of performing additional training (e.g., using the existing training dataset or another training dataset), modifying the model (e.g., changing the input features used by the model, changing the architecture of the model, or the like), or changing the training environment (e.g., changing training hyperparameters, changing a division of the training dataset into training, cross-validation, and holdout portions, or the like).

Consistent with disclosed embodiments, as described herein, training engine 160 can be configured to determine performance metric values for a model using data obtained in different contexts. Such performance metric values can be displayed to a user through user device 180. The user can then interact through user device 180 with training engine 160 to update the model, as described herein.

Consistent with disclosed embodiments, model trainer 161 can create or train predictive models. Model trainer 161 can create or train predictive models as instructed by training engine 160. For example, training engine 160 can instruct model trainer 161 to create and train a support vector machine using a training portion of a training dataset. Model trainer 161 can then create and train this support vector machine, returning the trained support vector to the training engine 160. As an additional example, training engine 160 can instruct model trainer 161 to create and train a penalized regression model. The training engine can configure model trainer 161 with a type of the penalized regression model (e.g., ridge regression, lasso regression, elastic net, or another suitable type), parameter values for the penalized regression (e.g., a lambda value that weights the sum of squared coefficient values), and a training portion of a training dataset. Model trainer 161 can then create and train the penalized regression model, returning the trained penalized regression model to the training engine 160. As an additional example, training engine 160 can instruct model trainer 161 to train a random forest model. Training engine 160 can provide hyperparameters, such as the size of each bootstrap sample, the number of features to consider at each split, the depth of each decision tree, and the number of decision trees in the random forest. Training engine 160 can provide a training portion of the training dataset. Model trainer 161 can then create and train the random forest model, returning the trained random forest model to the training engine 160.

Consistent with disclosed embodiments, model evaluation 163 can evaluate models trained by model trainer 161. Training engine 160 can provide model evaluation 163 a model and a cross-validation or holdout portion of the training dataset. In some embodiments, training engine 160 can specify one or more performance metrics for evaluation by model evaluation 163. In various embodiments, model evaluation 163 can be configured with a predetermined or default set of performance metrics. In some embodiments, the performance metrics can include confusion matrices, mean-squared-error, mean-absolute-error, sensitivity or selectivity, receiver operating characteristic curves or area under such curves, precision and recall, F-measure, or any other suitable performance metric.

Consistent with disclosed embodiments, prediction engine 170 can be configured to predict health information using a patient dataset and a trained predictive model. In some embodiments, prediction engine 170 can obtain the trained predictive model from model storage 130. In some embodiments, prediction engine 170 can obtain the patient dataset from data storage 140. In some embodiment prediction engine 170 can obtain the patient dataset (or a portion thereof) from another data storage location. This alternative data storage location can be associated with another entity or user. For example, prediction engine 170 can receive or retrieve the patient dataset from a healthcare system separate from the entity that controls prediction engine 170. In some embodiments, prediction engine 170 can obtain the model or data using data/model selector 150.

Consistent with disclosed embodiments, prediction engine 170 can apply the patient dataset to the trained predictive model to predict health information for the patient. The health information (or an indication thereof) can be provided by prediction engine 170 to user device 180. The health information can be stored on a computing device associated with pipeline 100 or provided to another system.

Consistent with disclosed embodiments, user device 180 can provide a user interface for interacting with other components of pipeline 100. The user interface can be a graphical user interface. The user interface can enable a user to configure data input engine 110 to extract, transform, and load data according to user specification. The user interface can enable the user to specify how the transformed data received by dataset generator 120 is converted into labeled training data (or patient data suitable for predictions). In some embodiments, the user interface can enable the user to interact with dataset generator 120 to manually or semi-manually label or annotate the training data. In some embodiments, the user interface can enable a user to interact with data/model selector 150 to manage data or models stored in model storage 130 or data storage 140. Such management can include deleting or creating models, deleting datasets, or restricting access by training engine 160 or prediction engine 170 to models or datasets. In some embodiments, the user interface can enable a user to interact with data/model selector 150 to push data or models to training engine 160 for training, or to prediction engine 170 for prediction. In some embodiments, the user interface can enable a user to interact with training engine 160 to create or select a predictive model for training, create or select a dataset for use in training the model, or select training parameters or hyperparameters. In some embodiments, the user interface can enable a user to interact with training engine 160 to display information related to training of the model (e.g., performance metric values, a change in loss function values during training, or other training information). In some embodiments, the user interface can enable a user to interact with prediction engine 170 to select a training model and patient data for use in predicting health information. In some embodiments, the user interface can enable a user to interact with prediction engine 170 to display the health information, store the health information on a computing device, or transmit the health information to another system.

Components of pipeline 100 can be implemented using one or more computing devices. Such computing devices can include tablets, laptops, desktops, workstations, computing clusters, or cloud computing platforms. In some embodiments, components of pipeline 100 can be implemented using cloud computing platforms. For example, one or more of data input engine 110, dataset generator 120, data/model selector 150, training engine 160, and prediction engine 170 can be implemented on a cloud computing platform. In some embodiments, components of pipeline 100 can be implemented using on-premises systems. For example, measurement system 101, records 103, or user device 180 can be, or be hosted on, on-premises systems. As an additional example, model storage 130 or data storage 140 can be, or be hosted on, on-premises systems.

Components of pipeline 100 can communicate using any suitable method. In some embodiments, two or more components of pipeline 100 can be implemented as microservices or web services. Such components can communicate using messages transmitted on a computer network. The messages can be implemented using SOAP, XML, HTTP, JSON, RCP, or any other suitable format. In some embodiments, two or more components of pipeline 100 can be implemented as software, hardware, or combined software/hardware modules. Such components can communicate using data or instructions written to or read from a memory (e.g., a shared memory), function calls, or any other suitable communication method.

As may be appreciated, the particular structure of pipeline 100 is not intended to be limiting. Consistent with disclosed embodiments, any two or more of record(s) 103, model storage 130, or data storage 140 can be combined, or hosted on the same computing device. Consistent with disclosed embodiments, data input engine 110 and dataset generator 120 can be omitted from pipeline 100. In such embodiments, datasets formatted and configured for use by training engine 160 or prediction engine 170 can be deposited in data storage 140 by another system or using another method. Consistent with disclosed embodiments, data input engine 110 and dataset generator 120 can be combined. In such embodiments, data extraction, transformation, and loading can be combined with feature extraction, annotation, and classification. Consistent with disclosed embodiments, data/model selector 150 can be combined with one or more of training engine 160 and prediction engine 170. For example, training engine 160 or prediction engine 170 can include functionality for retrieving selected data or models from model storage 130 or data storage 140.

Though shown with one user device 180, could have multiple user devices. Different user devices could be associated with different entities or different users having different roles. For example, user device 180 could be associated with a software engineer or data scientist who is developing the test, while another user device could be associated with a clinician who is using the test.

User device 180 can be combined with one or more other components of pipeline 100. In some embodiments, user device 180 and at least one of data/model selector 150, training engine 160, or prediction engine 170 can be implemented by the same computing device. In various embodiments, user device 180 and at least one of model storage 130 or data storage 140 can be implemented by the same computing device.

As may be appreciated, pipeline 100 can be integrated into a method for treating patients with a particular health condition. Prediction engine 170 can use a trained predictive model and input data obtained from a patient sample to determine a risk of the patient for experiencing a negative health outcome (e.g., a cardiovascular event, or recurrent cardiovascular event, within the next 4 years; dementia within the next 20 years; death within one year with stable heart failure reduced ejection fraction or heart failure preserved ejection fraction; or the like). If the patient has a risk greater than (or potentially equal to) a health-outcome dependent threshold, then the patient can be treated or monitored according to a first, more-aggressive or intensive protocol. If the patient has a risk less than (or potentially equal to) the health-outcome dependent threshold, then the patient can be treated or monitored according to a second, less-aggressive or intensive protocol.

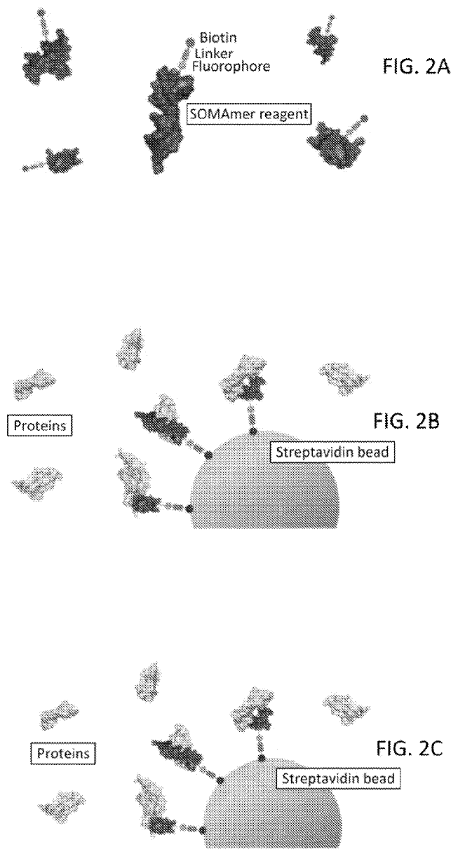

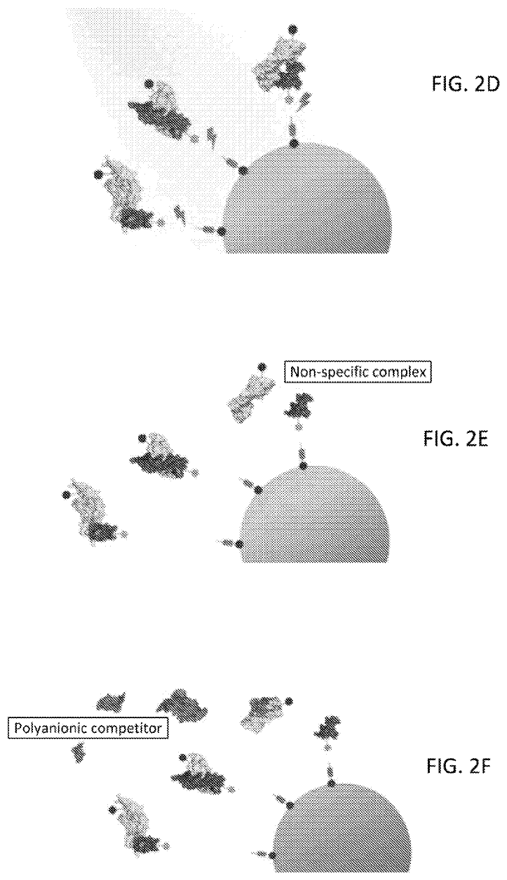

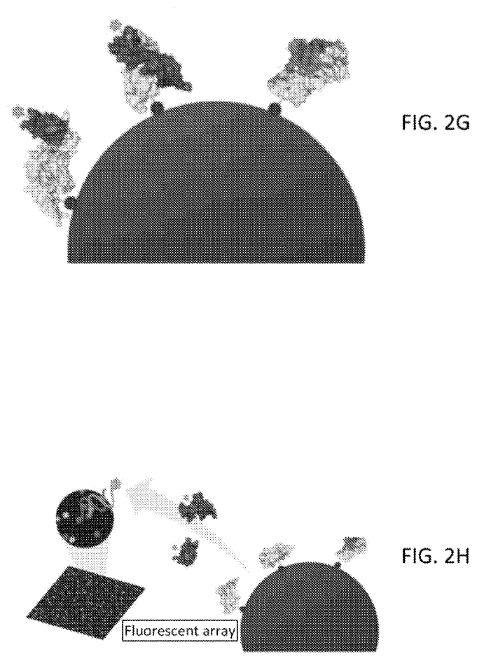

FIGS. 2A-2H provide a high-level depiction of stages in an exemplary aptamer-based serum or plasma assay 200, consistent with disclosed embodiments. Assay 200 can generate suitable input data for training a predictive model or predicting health information using a trained predictive model. Assay 200 can be performed using, at least in part, a testing system, such as measurement system 101 of pipeline 100.

Consistent with disclosed embodiments, assay 200 can quantitatively transform protein epitope availability in a biological sample into a specific DNA signal. In general, assay 200 can use SOMAmer® (Slow Off-rate Modified Aptamer) reagents that comprise short, single-stranded DNA sequences that incorporate hydrophobic modifications. Assay 200 can measure native proteins in complex matrices by transforming available binding sites on individual proteins into a corresponding SOMAmer reagent concentration, which can then be quantified by hybridization to microarrays. In this manner, the test takes advantage of SOMAmer reagents' dual nature as both protein affinity-binding reagents with defined three-dimensional structures and unique nucleotide sequences recognizable by specific DNA hybridization probes. Thus relative epitope concentrations can be converted into measurable nucleic acid signals that can be quantified using DNA-hybridization microarrays.

Consistent with disclosed embodiments, suitable versions of assay 200 can quantify relative levels of proteins in plasma spanning 10 logs in abundance. Such test versions can measure up to one thousand, three thousand, five thousand, seven thousand, ten thousand, or more unique protein analytes. Test can be performed on small volume samples (e.g., samples greater than 10 microliters, 20 microliters, 40 microliters, 100 microliters, 200 microliters, 400 microliter, 1 milliliter, or greater).

As may be appreciated, SOMAmer reagents can be selected against proteins in their native folded conformations. Thus such reagents may require an intact, tertiary protein structure for binding. Accordingly, unfolded and denatured—and therefore presumably inactive—proteins may not be detected by SOMAmer reagents (or may be detected with reduced or varying sensitivity).

As depicted in FIG. 2A, SOMAmer reagents can be synthesized with a fluorophore, photocleaveable linker, and biotin. Next, as depicted in FIG. 2B, SOMAmer reagents bound to streptavidin beads can be used to capture proteins from a complex mixture of proteins in a biological sample (e.g., a serum or plasma sample). Next, as depicted in FIG. 2C, unbound proteins can be washed away, and bound proteins can be tagged with biotin. Next, as depicted in FIG. 2D, electromagnetic radiation (e.g., ultraviolet light, or the like) can be applied to the solution to break the photocleaveable linker, releasing the proteins complexes and bound SOMAmers back into solution. As shown in FIG. 2E, non-specific complexes can disassociate from corresponding SOMAmers, while specific complexes remain bound. Next, as depicted in FIG. 2F a polyanionic competitor can be added to the solution. The polyanionic competitor can prevent rebinding of non-specific complexes. As shown in FIG. 2G, the biotinylated proteins (and bound SOMAmer reagents) can then be captured on streptavidin beads. The beads and bound proteins can be separated from the solution or concentrated. Next, as depicted in FIG. 2H, the SOMAmer reagents can be released from the protein complexes by denaturing the proteins. Fluorophores can be measured after hybridization to complementary sequences on a microarray chip. The fluorescence intensity detected on the microarray can be related to the amount of available epitope in the original sample.

As may be appreciated, assay 200 is intended to be exemplary. The disclosed system and methods are not limited to tests having these particular steps. In some embodiments, other aptamers (or even other classes of components) can be used to bind protein complexes. Alternative methods of inhibiting non-specific binding may be used in place of a polyanionic competitor. Alternative methods of separating protein-compound complexes may be used in place of capturing protein-compound complexes on streptavidin beads. Alternative indicia of protein levels can be measured in place of fluorophore measurements on a microarray chip. However, such alternative methods can still exhibit the technical challenges described herein. Therefore, such alternative systems and methods can benefit from the disclosed technical solutions.

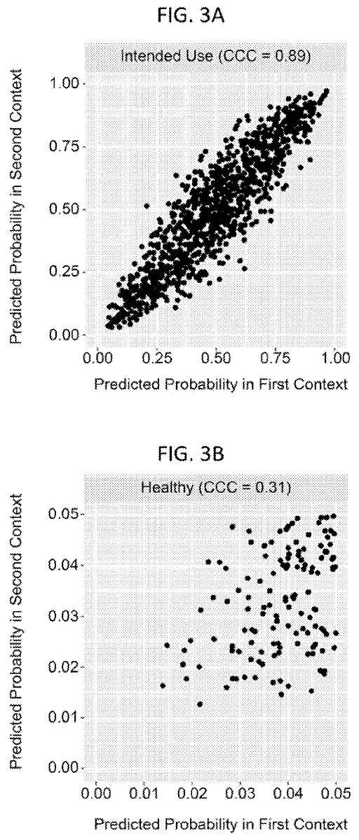

FIGS. 3A and 3B depict an exemplary challenge arising in the validation of proteomics models for predicting health information using aptamer-based serum or plasma tests, consistent with disclosed embodiments. In some embodiments, this technical challenge can be in ensuring concordant predictions across contexts. Such a challenge can arise when a proteomic model developed with input data acquired in a first context must be validated for use with input data acquired in another context.

Consistent with disclosed embodiments, contextual differences can include differences between samples, differences in sample handling, or differences in assay protocol. Consistent with disclosed embodiments, differences between samples can include the presence or absence of an interference agent in the sample, the use of citrate plasma versus EDTA plasma, the use of serum versus plasma, the fed/fasted state of the patient providing the sample, or other variations in sample characteristics that could affect the validity of a proteomics predictive model. The disclosed embodiments are not limited to any particular interference agents. In various embodiments, the interference agents can include non-steroidal anti-inflammatory drugs (NSAIDs), birth control medications, blood pressure medications, mental health medications (e.g., anti-depressants, antipsychotics, anxiolytics or hypnotics, mood stabilizers, stimulants, or the like), cholesterol medications, asthma medications, diabetes medications, thyroid medications, anti-viral or antibacterial medications, or other commonly used medications. Consistent with disclosed embodiments, differences in the sample handling can include differences in time between sample collection and sample freezing, temperature of sample freezing, duration of time at freezing temperature, number of freeze/thaw cycles for the sample, the time to spin a plasma sample, the time to clot or decant a serum sample, or other variations in sample handling conditions that could affect the validity of a proteomics predictive model. Consistent with disclosed embodiments, differences in assay protocol can include differences in reagents used, different dilutions of the same reagents, the addition or subtraction of steps in the assay protocol, or differences in the devices used to perform the assay protocol. For example, a first version of the assay protocol can be adapted to detect concentrations of 5000 proteins, while a second version of the assay protocol can be adapted to detect concentrations of 7000 proteins. These two versions of the assay protocol can use differing sets of aptamers, differing microarray chips, and potentially differing microarray scanners.

Consistent with disclosed embodiments, suitable input data acquired in the second context may be unavailable. The original samples used to generate the original input data may be missing, depleted, or degraded by the passage of time. Furthermore, presently available samples may differ from the original samples. For example, the original samples may have included samples from patients having a health condition that the predictive proteomic model was developed to detect. For example, the original samples may have been acquired over many years through collaboration with a medical center specializing the treatment of patients having the conditions that the predictive proteomic model was developed to detect. However, such patients may be extremely rare in the general population. And the presently available samples may be acquired from the general population. For example, the presently available samples may be acquired from routine blood draws at a community health center. Therefore the presently available samples are unlikely to include patients having the condition that the predictive proteomic model was developed to detect.

FIG. 3A depicts an exemplary correlation between the outputs of a predictive proteomic model for input data acquired using two different assay protocols. In this toy example, the output represents a risk of death within a year for people having a rare disease. The two different assay protocols are a 5000-protein protocol and 7000-protein protocol. The 7000-protein protocol includes the 5000 proteins and 2000 additional proteins. The predictive proteomic model was developed using input data acquired according to the 5000-protein protocol. Input data acquired using the 7000-protein protocol can be input to the predictive proteomic model by truncating the input to include only the shared 5000 proteins.

In this example, a set of samples taken from patients having the rare disease is available. Two sets of input data can be generated for each sample, one set according to the 5000-protein protocol and one set according to the 7000-protein protocol. These sets of input data can be applied to the predictive proteomic model to generate probabilities of death within a year. As apparent in FIG. 3A, these predicted probabilities are highly correlated, having a Lin's concordance correlation coefficient (CCC) of 0.89.

FIG. 3B depicts an exemplary correlation between the outputs of the same predictive proteomic model for the same 5000-protein and 7000-protein protocols. In this example, the set of samples taken from patients having the rare disease is unavailable. Instead, samples from healthy normal patients are used. As described with regards to FIG. 3A, two sets of input data can be generated for each sample, one set according to the 5000-protein protocol and one set according to the 7000-protein protocol. These sets of input data can be applied to the predictive proteomic model to generate probabilities of death within a year. As apparent in FIG. 3B, these predicted probabilities are not highly correlated, having a CCC of 0.31.

As may be appreciated, this lack of correlation arises from a severe restriction in the range of the predicted probabilities. The predictive proteomic model correctly finds that healthy normal patients are extremely unlikely to die from the disease within a year. But as a result, the range of predicted probabilities depicted in FIG. 3B is approximately twenty times smaller than the range of predicted probabilities depicted in FIG. 3A. Thus naïve testing using the available set of samples will underestimate the reliability of the predictive proteomic model when used with the 7000-protein protocol.

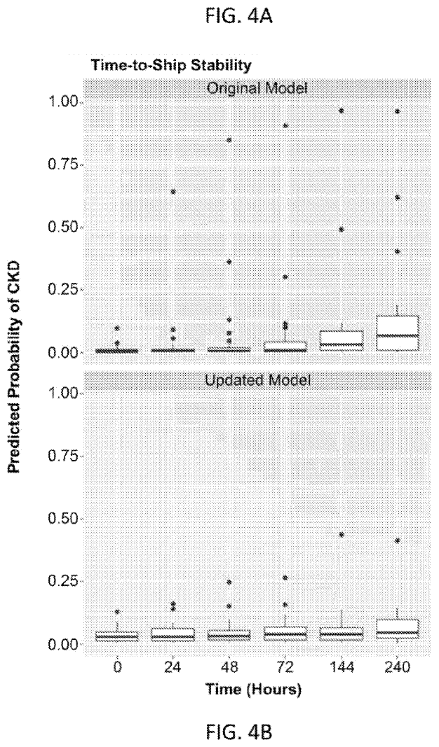

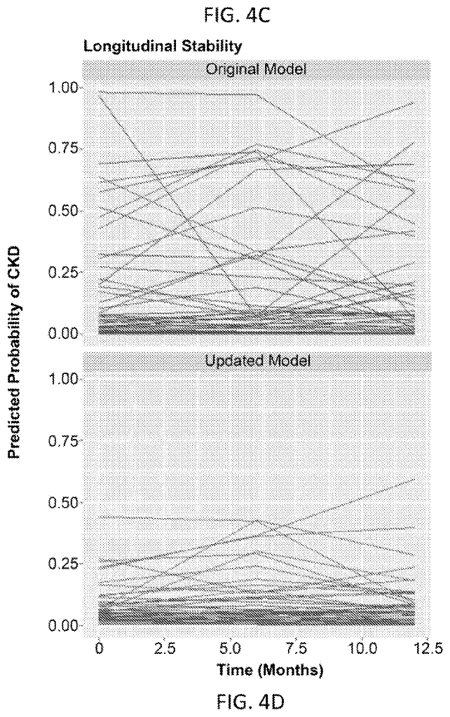

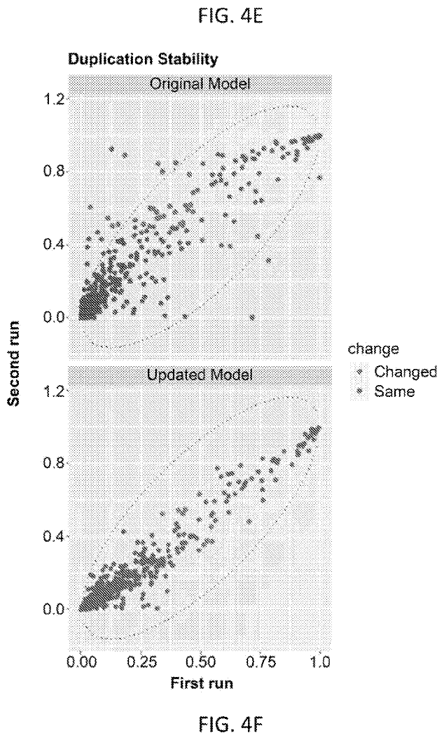

FIGS. 4A to 4F depict exemplary model-resilience challenges arising in the training of proteomics models for predicting health information using aptamer-based blood tests, consistent with disclosed embodiments. As may be appreciated from the description of an exemplary SOMAmer-based blood test with regards to FIGS. 2A to 2H, an aptamer-based blood test can be sensitive to sample-handling changes that may affect protein shapes or concentrations (e.g., through degradation over time, reactions with other sample components, or the like). Changes in protein levels will clearly affect the measured protein levels. Similarly, any denaturing of proteins will affect the ability of aptamer complexes to bind to such proteins, affecting the measured protein levels.

Accordingly, sample handling conditions can affect the output of a proteomics model that takes protein levels as inputs. The dependence of the predicted output on sample handling conditions can be independent of the predictive power of the model. For example, two proteomics models can be similarly predictive, but the first model may exhibit substantial variance with respect to a particular variation in sample handling, while the second model may be resilient to this particular variation. Such differences may arise from the particular proteins relied upon by the different models. For example, two proteins may provide similar or correlated information about a health condition. A parsimonious model may depend upon one of the proteins, but not both. But one of the two proteins may be much more stable than the other protein. Thus, even through the two proteins may be equivalent from a predictive standpoint, a resilient model may be designed to depend upon the more-stable protein and eschew the less-stable protein.

FIGS. 4A and 4B depict a dependence on sample handling conditions of two proteomics models trained to predict the likelihood of having chronic kidney disease, consistent with disclosed embodiments. In this study, the variation in sample handling was in the time between sample collection and sample shipping on dry ice to the testing laboratory. Multiple healthy normal samples were collected and six different times to between sample collection and shipping were investigated. For each sample, multiple aliquots were prepared, each aliquot corresponding to one of the six variations in time-to-ship. Two predictive models were trained.

FIG. 4A depicts the dependence of predicted likelihood of having chronic kidney disease on time-to-ship for a first proteomics model. An increase in the predicted likelihood of having chronic kidney disease is observed with increasing time-to-ship. There is both an increase in the median predicted likelihood and the appearance of significant outliers in the predicted value. As may be appreciated, a clinician may not indicate a delay in shipping a sample to a laboratory. The laboratory may therefore provide an incorrect prediction due to the dependence of predicted risk on time-to-ship.

FIG. 4B depicts the dependence of predicted likelihood of having chronic kidney disease on time-to-ship for a second proteomics model. This second proteomics model was developed by updating the first proteomics model based on the determined sensitivity of the first proteomics model to differences in time-to-ship. Contributions to the model from proteins that demonstrated sensitivity to differences in time-to-ship were reduced, as compared to the first model. As may be appreciated, the second predictive proteomic model exhibits less of an increase in the predicted risk than the first predictive proteomic model. Furthermore, the variability of predicted risk has decreased, a change that manifests in the plot as a slight increase in the interquartile range combined with a decrease in the number and extent of outliers.

In addition to changes arising from sample handling, assessment on external samples may identify issues with overfitting in the trained model. FIG. 4C depicts a variability of predicted risk of developing chronic kidney disease over time for a first proteomics model. In this study, the predicted risk was determined using repeated samples over time for the same patients. The samples were obtained at intervals over twelve months. As may be appreciated, patients that originally exhibited an elevated risk of developing chronic kidney disease should see that risk maintained throughout the testing interval. As observed in FIG. 4C, some patients exhibited dramatic changes in predicted risk over time, potentially due to overfitting in the original model, which then provided unstable predictions on an external datasets.

FIG. 4D depicts the variability of predicted relative risk of developing chronic kidney disease over time for a second proteomics model. The second model exhibits a reduced variance in the predicted risk. Such a model can be generated by identifying at least one protein that contributes to the first predictive model and exhibits a variation between the samples. The contribution of that protein to the first model can be reduced. In some embodiments, the first model can then be retrained to generate the second model.

In addition to changes arising from sample handling, measured protein levels may exhibit process variability. Some measured protein levels can exhibit greater measurement variability and cause overfitting in a model. A proteomics model that depends upon the protein level values of such proteins may exhibit greater prediction variability, even in across aliquots taken from the same sample, than a proteomics model that does not depend upon the protein level values of such proteins.

FIG. 4E depicts risk values determined using a first proteomics model for two different aliquots of the same sample. In this example, a disease diagnosis depends on the predicted risk values. The values shown as open circles were given different diagnosis for different aliquots of the sample (e.g., the upper left quadrant and lower right quadrants indicate a negative diagnosis for one aliquot and a positive diagnosis for another aliquot). The values shown as closed circles received the same diagnosis for both aliquots of the sample.

FIG. 4F depicts risk values determined using a second proteomics model for two different aliquots of the same sample. As compared to the predictions in FIG. 4E, far fewer samples exhibit differences in diagnosis between aliquots of the same sample. The second proteomics model can be generated from the first proteomics model by identifying proteins that contribute to the first proteomics model and exhibit high intra-aliquot variability and reducing the contribution of these proteins in generating the second proteomics model.

FIG. 5 depicts an exemplary process 500 for validating proteomics models, consistent with disclosed embodiments. The proteomics models can be validated against input data collecting using differing data collection protocols. Process 500 can be performed using a machine learning pipeline, such as pipeline 100 described above with regards to FIG. 1. For convenience of description, process 500 is described herein as being performed using training engine 160. However, process 500 is not limited to such an implementation. Consistent with disclosed embodiments, process 500 can be performed using other components of pipeline 100 or other machine learning systems. For example, process 500 can be performed using a stand-alone system or computing device for training machine learning models.

Process 500 can provide a technical solution to the technical problem described above with regards to FIGS. 3A and 3B. Consistent with disclosed embodiments, process 500 can enable validation of a proteomics model for use in a second context when that proteomics model was developed for use in a first context. In some embodiments, the input data used to develop the proteomics model may be available, the original samples from which this input data was generated may no longer be available. And available samples may not span the full range of the potential inputs to the model. For example, such available samples may be predominantly or entirely from “healthy normal” patients. Thus, the available samples may not support validation of the full range of proteomics model outputs.

Consistent with disclosed embodiments, process 500 can compensate for the unavailable original samples by using the available samples to determine a protein level noise. The protein level noise can characterize the difference in protein level measurements between the first context and the second context. The protein level noise can be used, together with the input data originally used to develop the proteomics model, to generate a validation dataset. The validation dataset can then be used to validate the proteomics model in the second context. Thus, even though the available samples do not span the full range of potential inputs to the model, these samples can still support validation of the full range of proteomics model outputs.

In step 510 of process 500, training engine 160 can obtain control, treatment, and training datasets, consistent with disclosed embodiments. In some embodiments, these datasets can include protein level measurements (e.g., measurements of relative or absolute protein levels in an aliquot of a sample). Such protein level measurements can be numeric.

For example, the datasets can include numeric values in relative fluorescence units (RFU) or fluorescence units (FLU). These values can indicate protein level. In some embodiments, the control and treatment datasets can be or include paired datasets. Such paired datasets can be generated using multiple aliquots from the same sample, or paired samples collected from the same patients. The control data can be generated using the first context, while the treatment dataset can be generated using the second context. The training dataset may have been generated using the original samples in the first context and should represent the same inputs as the data used to train the model.

In some embodiments, the first and second context can test for different sets of proteins. For example, the first context may be or include a previously developed assay, while the second context may be or include a next-generation assay. The next-generation assay can test for a superset of the proteins tested for in the previously developed assay. For example, the previously developed assay may test for 5000 proteins, while the next-generation assay may test for those 5000 proteins, plus an additional 2000 proteins.

In some embodiments, the first and second context can use different sample-handling techniques. For example, the control dataset can be generated from citrated plasma samples, while the treatment dataset can be generated from potassium ethylenediaminetetraacetic acid (EDTA) plasma samples. The citrated plasma samples and the EDTA plasma samples can be obtained from the same patients. As an additional example, the first and second context can use differing times from drawing blood to spinning the sample, different times from spinning the sample to decanting the sample, different times from decanting the sample to freezing the sample, different freezing durations or storage temperatures, different numbers of freeze/thaw cycles, of the like.

In some embodiments, the first and second context can use different assay protocols. For example, such differences can include differences in reagents used, different dilutions of the same reagents, the addition or subtraction of steps in the assay protocol, or differences in the devices used to perform the assay protocol. As an additional example, the first context can include a first set of aptamers and the second context can include a second set of aptamers. The second set can be, at least in part, revised versions of the first set of aptamers.

In some embodiments, the second context can include an interference agent absent from the first context. For example, preparing an aliquot according to the second context can include adding the interference agent to the aliquot. Thus process 500 can be used to determine whether the presence of a commonly used medication in the blood of a patient negatively affects the performance of the predictive proteomic model.

The disclosed embodiments are not limited to any particular method of obtaining the control, treatment, and training datasets. In some embodiments, training engine 160 can retrieve these datasets from data storage 140, or another location. In various embodiments, the training engine 160 can retrieve these datasets from another system (e.g., a healthcare system, insurance system, or the like). In some embodiments, the control, treatment, and training datasets can be generated from aliquots or samples using a proteomics assay, such as assay 200. However, the disclosed embodiments are not limited to embodiments that use assay 200. Other proteomics assays, or other types of assays, can additionally or alternatively be used to generate control, treatment, and training datasets that can be used to validate predictive models, consistent with disclosed embodiments.

In step 520 of process 500, training engine 160 can estimate protein level noise, consistent with disclosed embodiments. In some embodiments, protein level noise can be estimated on a per-protein basis. Training engine 160 (or another component of pipeline 100, such as dataset generator 120, or the like) can determine pairwise differences between protein level measurements for the treatment dataset and corresponding protein level measurements for the control dataset. For example, a first entry in the control dataset and a second entry in the treatment data can correspond to aliquots drawn from the same sample. The first entry and the second entry can include protein level measurements for the same proteins. Training engine 160 can subtract the protein level measurements from the first entry from the protein level measurements for the second entry, to determine the pairwise differences for each protein. Training engine 160 can determine such pairwise differences for each entry in the control dataset and each corresponding entry in the treatment dataset.

Consistent with disclosed embodiments, training engine 160 can characterize, for each protein, a distribution of pairwise differences. Characterizing the distribution can include estimating the distribution of pairwise differences (e.g., using a histogram of pairwise differences, estimating parameters of a parametric model of the pairwise differences, or the like), estimating statistics (e.g., mean, median, mode, standard deviation, quartiles, percentiles, or the like) or moments (e.g., first moment, second moment, third moment, or the like) of the distribution of pairwise differences, maintaining the set of pairwise differences and resampling from that set, or other suitable methods of characterizing the distribution of pairwise differences.

As may be appreciated, depending on the differences between the first context and the second context, the control and treatment datasets may include protein level measurements for differing sets of proteins. For example, the treatment dataset may include protein level measurements for a superset of the proteins in the control dataset. In some embodiments, pairwise differences can be determined and distributions characterized for proteins present in both databases.

In step 530 of process 500, training engine 160 can generate a validation dataset, consistent with disclosed embodiments. The validation dataset can include adjusted protein level values. The adjusted protein level values can be generated using the estimated protein level noise and the training dataset. In some embodiments, the validation dataset can include multiple entries. Each entry can correspond to an entry in the training dataset. In some embodiments, training engine 160 can generate adjusted protein level measurements for each protein in each entry.

Consistent with disclosed embodiments, an adjusted protein level value can be a function (e.g., a sum, or the like) of a protein level value in the training dataset and a noise sample. In some embodiments, training engine 160 can generate the noise sample. Generation of the sample can depend on the characterization of the distribution in step 520. In some embodiments, when the shape of the distribution was estimated (e.g., using a histogram), a distribution having the estimated shape can be sampled. In some embodiments, when statistics or moments were estimated, the statistics or moments can be used to generate the sample. For example, when the mean and standard deviation are estimated for a protein in the training dataset, a normal distribution having the estimated mean and standard deviation can be sampled to generate the sample for that protein. In some embodiments, the set of pairwise differences for a protein can be resampled to generate the sample for the protein.

In step 540 of process 500, training engine 160 can generate a set of predictions by applying the validation dataset to a proteomics model, consistent with disclosed embodiments. The proteomics model can be the model developed using the training dataset. Each entry in the validation dataset can be used to generate a corresponding prediction in the set of predictions. How the validation dataset is applied to the proteomics model to generate the set of predictions can depend on the implementation of the proteomics model. In some embodiments, a proteomics model can be implemented as an instance of an object. The object can specify methods for training the model and for making predictions using the model. Generating a prediction using the model can then involve invoking the method with an entry as the input. For example, suppose that the function svm.svc(parameters) returns a support vector machine object having certain parameters. This object can have a fit(x, y) method that trains the data (using x and y training data) and a predict(x) method that takes a vector of samples (each including a vector of features) generates a vector of class labels. In this simple example, generation, training, and prediction can be:
proteomics_model=svm.svc(parameters);
proteomics_model.fit(x_training, y_training); and
classifications=proteomics_model.predict(x_test).

As an additional example, the function linear_model.LogisticRegression(parameters) can return penalized logistic regression object having certain parameters. As with the support vector machine object described above, the object can specify a fit( ) method for training the object and a predict( ) method for generating predictions using input data.

As may be appreciated, the above examples of support vector machine and penalized logistic regression objects are exemplary and not intended to be limiting. The particular manner in which the validation dataset is applied to the proteomics model will depend on the particular implementation of the proteomics model.

In step 550 of process 500, training engine 160 can determine one or more performance measures for the proteomics model using the set of predictions generated in step 540. In some embodiments, training engine 160 can determine a performance measure that depends upon the agreement of two sets of predictions: an original set of predictions generated by the proteomics model using the training data and the set of predictions generated in step 540. In some embodiments, the performance measure can be Lin's concordance correlation coefficient, Pearson's correlation coefficient, or another suitable performance measure. In some embodiments, training engine 160 can determine a performance measure dependent upon an agreement between a ground truth associated with the training data and the set of predictions generated in step 540. In some embodiments, the ground truth can be specified by labels associated with the entries in the training dataset. Such labels could specify the presence or absence of a condition, a binned survival time, or another suitable ground truth concerning health information for a patient.

Consistent with disclosed embodiments, training engine 160 can determine a validity indication based on the one or more performance measures. In some embodiments, the validity indication can be the value(s) of the one or more performance measures. In various embodiments, the validity indication can depend upon the value(s) of the one or more performance measures. For example, a value of a performance measure can be binned or thresholded, with values in a certain bin or below a certain threshold assigned a “warning” or “failed” indicator, and values in another bin or above another threshold assigned a “passed” or “valid” indicator.

Consistent with disclosed embodiments, process 500 can include an optional operation, not shown in FIG. 5. This operation can be performed by training engine 160 after obtaining the control and treatment datasets (e.g., in step 510). In some embodiments, training engine 160 can determine a pairwise difference between a control set of predictions generated by applying the control dataset to the proteomics model and a treatment set of predictions generated by applying the treatment dataset to the proteomics model. As may be appreciated, when the control and treatment datasets include overlapping sets of proteins, the pairwise difference may only be calculated for proteins in the intersection of the sets. In some embodiments, training engine 160 can determine a statistic (e.g., a mean, median, 75th percentile, 90th percentile, or other suitable threshold) of the pairwise differences. In some embodiments, training engine 160 can determine whether the statistic satisfies an invalidity condition. The invalidity condition can depend on a characteristic of assay 200. For example, the predictive model can have an inherent variability arising from noise inherent in assay 200 (e.g., protein level noise determined based on differences in test-retest values for protein levels, or the like).

In some embodiments, the invalidity condition can be at least in part satisfied when the statistic of the pairwise differences exceeds a function of the statistic of inherent variability. For example, the statistic of inherent variability can be the standard deviation o of this inherent variability. The function of the statistic can be a multiple (e.g., a multiple selected in the range from 0.5 to 5, such as 3 or another suitable value) of the statistic. In some embodiments, the invalidity condition can be satisfied when the statistic of the pairwise differences exceeds a multiple of the standard deviation of the inherent variability. In some embodiments, satisfaction of the invalidity condition can further require that a statistic test (e.g., a paired t-test or the like) reject a null hypothesis that the statistic of the pairwise differences equals zero.

Consistent with disclosed embodiments, if the invalidity condition is satisfied, then process 500 can proceed to step 520, otherwise, process 500 can terminate.

FIG. 6 depicts an exemplary process 600 for developing proteomics models, consistent with disclosed embodiments. The development of the proteomics models can be structured to enhance the resilience of such models to variations in input data. As described herein, input datasets can be generated in different contexts, and variations in input data can arise from differences between such contexts. Process 600 can be performed using a machine learning pipeline, such as the pipeline described above with regards to FIG. 1. For convenience of description, process 600 is described herein as being performed using training engine 160. However, process 600 is not limited to such an implementation. Consistent with disclosed embodiments, process 600 can be performed using other components of pipeline 100 or other machine learning systems. For example, process 600 can be performed using a stand-alone system or computing device for training machine learning models.

Process 600 can provide a technical solution to the technical problem described above with regards to FIGS. 4A to 4F. Consistent with disclosed embodiments, process 600 can enable development of a proteomics model that is resilient to differences in the context in which input datasets were obtained. As may be appreciated, the training data used to develop the proteomics model development may be obtained from samples handled strictly in accordance with sample-handling guidelines. But samples received from clinicians, healthcare systems, laboratories, or other users may not have been handled in such strict accordance with sample-handling guidelines. Consistent with disclosed embodiments, the proteomics model may therefore be developed to be resilient to variations in sample-handling. Furthermore, the proteomics model may be developed to be resilient to variations in the assay process, or in the devices used to measure protein levels.

In some embodiments, process 600 can be combined with process 500, described above. For example, the proteomics model may have been developed using samples that are no longer available. And testing resilience of the proteomics model may require many samples obtained in many varying contexts. Process 500 can enable the training data used to develop the proteomics model to be used for resilience testing. Applying process 500, the control dataset can be obtained using the same context as the training data, while multiple treatment datasets can be obtained using variations in the context in which the data is acquired. The effect of these variations can then be investigated by estimating protein level noise and generating validation datasets corresponding to each of the treatment datasets.

In some embodiments, process 600 can be performed as part of an iterative process of training and development. For example, a user may interact with training engine 160 (e.g., through user device 180) to select and train a proteomics model. The user may then interact with training engine 160 to repeatedly perform process 600 until a satisfactory proteomics model is developed. This model may exhibit a desired degree of resilience across a determined set of contextual variations.

In step 610 of process 600, training engine 160 can obtain a proteomics model, consistent with disclosed embodiments. The proteomics model may have been trained (e.g., by training engine 160 or another system) to generate a prediction based on protein level measurements. The proteomics model may have been trained using a training dataset obtained from training samples in a first context. Training engine 160 can obtain the proteomics model from a model storage, such as a model storage 130, or another suitable storage location.

In step 620 of process 600, training engine 160 can obtain treatment datasets including protein level measurements, consistent with disclosed embodiments. In some embodiments, the treatment datasets can be generated by assaying treatment samples obtained in treatment contexts. In some embodiments, each treatment context can differ along one or more dimensions. For example, the dimension can be spin time and the treatment contexts can have spin times of 0.5, 1.5, 3, 9, and 24 hours. As an additional example, the dimension can be number of freeze-thaw cycles and the treatment contexts can have 2, 3, 4, 5, and 10 freeze-thaw cycles. As described herein, the treatment datasets can be generated by pipeline 100 from the treatment samples using an assay, such as assay 200, and a measurement system, such as measurement system 101. Alternatively or additionally, treatment datasets can be obtained by pipeline 100 (or training engine 160) from another system.

In some embodiments, each treatment dataset can be applied to the proteomics model to generate a set of predictions. These predictions can then be analyzed in step 630 of process 600. In various embodiments, a control dataset can also be obtained in step 620 of process 600. The control dataset can be generated from a sample obtained in the same context as the sample used to generate the training dataset (e.g., the dataset used to train the proteomics model). As described with regards to process 500, the control dataset, the training dataset, and each treatment dataset can be used to generate validation datasets, each validation dataset corresponding to and generated using a treatment dataset.

In step 630 of process 600, a dependence of the proteomics model on the differences between the second contexts can be determined, consistent with disclose embodiments. In some embodiments, the dependence can be determined based on one or more performance measures for the proteomics model. In various embodiments, the dependence can be determined based on displayed indica concerning the sets of predictions generated in step 620.

Consistent with disclosed embodiments, training engine 160 can determine the one or more performance measures for the proteomics model. Training engine 160 can determine the one or more performance measures for one or more of the treatment (or validation) datasets. In some embodiments, training engine 160 can determine the one or more performance measures for the control (or training) datasets. In some embodiments, training engine 160 can determine a performance measure dependent on an agreement between predictions generated by applying the control dataset (or the training dataset) to the proteomics model and predictions generated by applying a treatment dataset (or a validation dataset) to the proteomics model. In some embodiments, training engine 160 can determine a performance measure dependent on an agreement between a ground truth and predictions generated by applying a control or treatment dataset (or a training or validation dataset) to the proteomics model.

Consistent with disclosed embodiments, training engine 160 can provide a user interface accessible through user device 180. The user interface can enable training engine 160 to display the values of the one or more performance metrics. In some embodiments, the user interface can display boxplots, q-q plots, histograms, tables, or other suitable displays of the sets of predictions generated in step 620. Examples of such displays include FIGS. 4A to 4F, 7A to 7D, 8A to 8D, and 9A to 9B.

Consistent with disclosed embodiments, the dependence of the proteomics model on the differences between the second contexts can be determined automatically. In some embodiments, training engine 160 can automatically determine whether the one or more performance measures differ between the predictions generated using the control and treatment datasets (or between the training and validation datasets). In some embodiments, training engine 160 can automatically determine whether such differences are statistically significant. In various embodiments, a user can determine the dependence based on a display of the values of the one or more performance metrics. In some embodiments, the dependence of the proteomics model can be determined semi-automatically. Training engine 160 can automatically determine the values of the one or more performance measures, and a user can determine the dependence based on a display of the values of the one or more performance measures. In some embodiments, the dependence of the proteomics model can be determined manually. Training engine 160 can provide for display the sets of predictions generated in step 620, and the user can determine the dependence based on this display.

In step 640 of process 600, the proteomics model can be updated based on the dependence determined in step 630, consistent with disclosed embodiments. In some embodiments, a protein in the input datasets applied to the proteomics model can be identified. The proteomics model can then be updated in a manner that reduces the importance of the protein in the proteomics model.

Consistent with disclosed embodiments, the protein can be identified based on an effect of the protein on the variability of the predictive model between control and treatment datasets, or between the training and validation datasets (e.g., a “variability effect”). The manner of the effect and the manner of identification can depend on the implementation of the proteomics model. In some embodiments, the variability effect can depend on an importance of the protein to the model. For example, the importance of the protein to a regression proteomics model can depend on the magnitude of a coefficient of the protein in the regression proteomics model. As further example, the importance of the protein to an SVM proteomics model can depend on a feature weight of the protein in the SVM proteomics model. As further example, the importance of the protein to a random forest model can be computed using Gini importance, mean decrease accuracy, permutation-based importance, Shapley value-based importance, or any other suitable method. In some embodiments, the variability effect can depend on a variance of the protein level between the control and treatment datasets (or between the training and validation datasets). In some instances, the greater this variance in measured protein levels, the greater the effect of the protein on the variability of the predictive model.

Consistent with disclosed embodiments, the proteomics model can be updated to reduce the variability effect of an identified protein. The variability effect can be reduced by decreasing a magnitude of the weight or coefficient associated with the protein, excluding the protein from the input dataset, and retraining the model, or another suitable manner. In some embodiments, training engine 160 can automatically update the proteomics model. For example, training engine 160 can automatically identify the proteins with the greatest variability effect. Training engine 160 can then update the model to reduce the variability effect of one or more of these proteins. In some embodiments, training engine 160 can semi-automatically update the proteomics model. For example, training engine 160 can automatically identify the proteins with the greatest variability effect and display indicia of these proteins to the user. The user can then select zero, one, or more of the identified proteins. Training engine 160 can then update the proteomics model to reduce the variability effect of any selected protein(s). In some embodiments, the user can interact with a user interface provided by training engine 160 to identify the proteins with the greatest variability effect. For example, the user can inspect model weights or coefficients, model architecture, or the calculated contributions of different proteins to the output of the model. The user can then provide commands to training engine 160 to update the proteomics model to reduce the variability effect of any identified protein(s).

As may be appreciated, the identification of proteins may involve more than merely removing the proteins with the greatest variability effect from the model. In some instances, such proteins may provide information necessary to the functioning of the proteomics model. In such circumstances, rather than removing a protein, a user can determine the difference in contexts between the control and treatment datasets be identified as potentially harmful to the performance of the proteomics model. For example, when delaying clotting of serum affects the output of the model, and removing the proteins responsible for the effect would unduly compromise the performance of the model, instructions for collecting samples could be updated to emphasize that clotting of serum be performed according to the instructions and without delay.

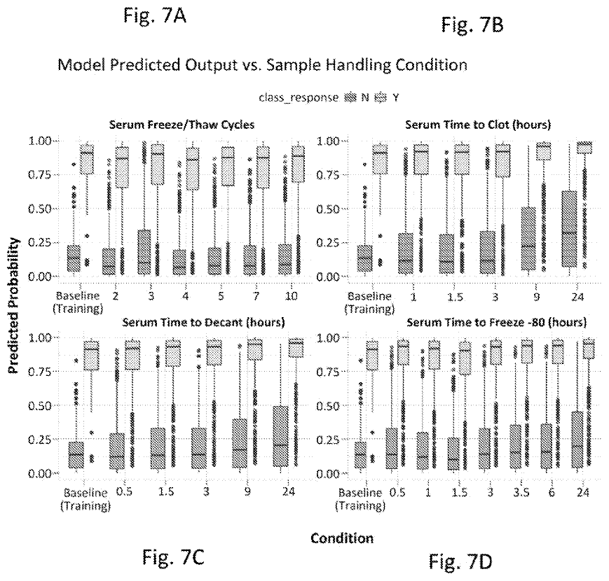

FIGS. 7A to 7D depict the effect of differences in sample handling on the output of a proteomics model, consistent with disclosed embodiments. In this example, the samples were serum samples. Sample handling was varied across four dimensions: number of freeze/thaw cycles, serum time to clot, serum time to decant, and serum time to freeze (at −80 C). A proteomics model was developed using the training data. In this example, the proteomics model was trained using known significant aptamers plus five randomly selected aptamers. Consistent with process 500 and steps 610 to 630 of process 600, predictions were generated using the training dataset, and control and treatment datasets. Boxplots of these predictions, broken out by class label, are displayed in FIGS. 7A to 7D. As may be appreciated, a proteomics model may show stable or monotonic change across variations in each of the four dimensions. Should updating the model to exhibit such stability or monotonic change across a dimension of sample handling unduly decrease the performance of the model, sample instructions for collection can emphasize the importance of this dimension of sample handling.

FIG. 7A depicts a dependence of a predicted probability value, generated by applying an entry in a training or validation dataset to a proteomics model, on variations in a number of freeze-thaw cycles for the sample. As can be observed, a number of outlier observations increases rapidly between the baseline sample handling value and two freeze-thaw cycles, remaining high thereafter as the number of freeze thaw cycles increases.

FIG. 7B depicts a dependence of the predicted probability value on variations in the time to clot for the sample. As depicted, the median predicted probability value increases for the negative class observations as time to clot increases. The distribution of predicted probability values for the negative class observations also exhibits an increasing skew towards improper, higher values of the predicted probability. For the positive class observations, a number of outliers with improperly low predicted values increases as the time to clot increases.

FIG. 7C depicts a dependence of the predicted probability value on variations in the time to decant for the sample. As depicted, the median predicted probability value increases for the negative class observations as time to decant increases. The distribution of predicted probability values for the negative class observations also exhibits an increasing skew towards improper, higher values of the predicted probability. For the positive class observations, a number of outliers with improperly low predicted values increases as the time to decant increases.

FIG. 7D depicts a dependence of the predicted probability value on variations in the time to freeze at −80C for the sample. As depicted, increasing the time to freeze beyond the baseline value immediately affects the distribution of predicted probability values, increasing outliers for both the negative-and positive-class samples.

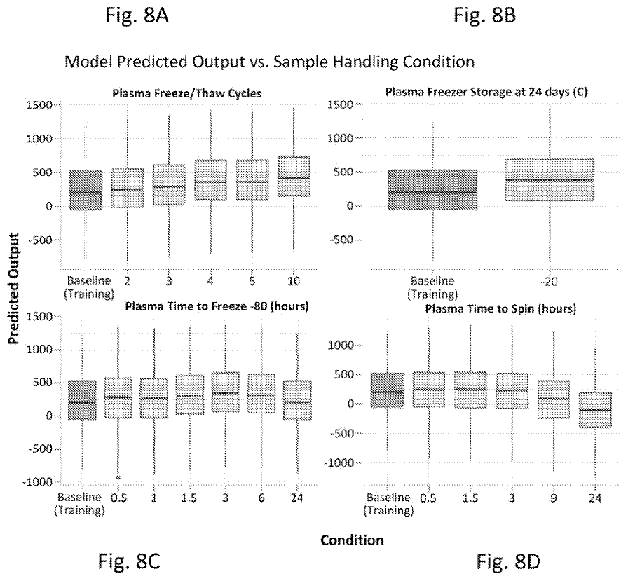

FIGS. 8A to 8D depict the effect of differences in sample handling on the output of a proteomics model, consistent with disclosed embodiments. In this example, the samples were plasma samples. Sample handling was varied across four dimensions: number of freeze/thaw cycles, plasma freezer temperature for 24 days storage, plasma time to freeze at −80 C, and plasma time to spin. A proteomics model was developed using the training data. In this example, the proteomics model was trained using known significant aptamers plus five randomly selected aptamers. Consistent with process 500 and steps 610 to 630 of process 600, predictions were generated using the training dataset, and control and treatment datasets. Boxplots of these predictions are displayed in FIGS. 8A to 8D.

FIG. 8A depicts a dependence of a predicted output value, generated by applying an entry in a training or validation dataset to the proteomics model, on variations in a number of freeze-thaw cycles for the sample. As can be observed, the median predicted output value increases with increasing number of freeze-thaw cycles.

FIG. 8B depicts a dependence of the predicted output value on variations in the plasma storage temperature (e.g., −80 C versus −20 C) for 24 days of storage of the sample. As shown, the predicted output increase when the plasma is stored at the higher temperature.

FIG. 8C depicts the predicted output value on variations in the time to freeze at −80 C for the sample. As depicted, there is not an obvious dependence of the predicted output value for these differing times to freeze.

FIG. 8D depicts a dependence of the predicted output value on variations in the time to spin the sample. As depicted, increasing the time to spin from baseline to nine hours, or more, affects the output value.

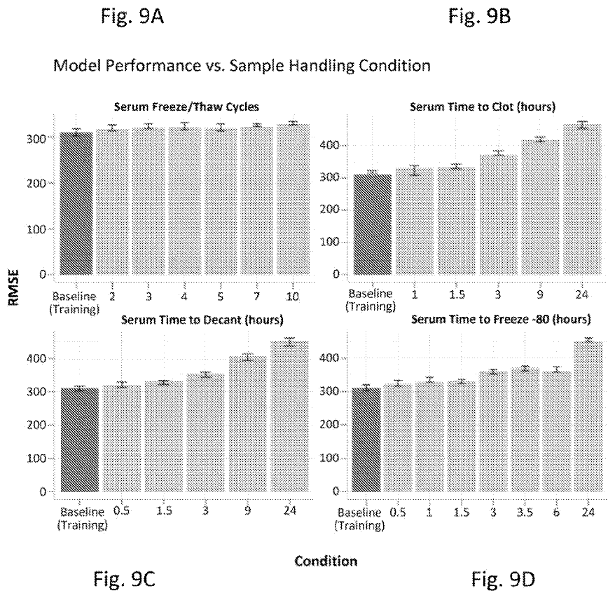

FIGS. 9A to 9D depict the effect of differences in sample handling on the predictive performance of predictions made by a proteomics model, consistent with disclosed embodiments. In this example, the predictive performance is the root-mean-squared-error (RMSE) and the samples were serum samples. Sample handling was varied across four dimensions: number of freeze/thaw cycles, serum time to clot, serum time to decant, and serum time to freeze (at −80 C). A proteomics model was developed using the training data. In this example, the proteomics model was trained using known significant aptamers plus five randomly selected aptamers. Consistent with process 500 and steps 610 to 630 of process 600, predictions were generated using the training dataset, and control and treatment datasets. Bar charts of the measured RMSE are displayed in FIGS. 9A to 9D.

FIG. 9A depicts a dependence of an RMSE value on variations in a number of freeze-thaw cycles for the sample. As can be observed, the RMSE value did not increase significantly between 1 and 10 freeze-thaw cycles.

FIG. 9B depicts a dependence of an RMSE value on variations in the time to clot for the sample. As can be observed, the RMSE value increase significantly between the baseline time to clot and times greater than 3 hours.

FIG. 9C depicts a dependence of an RMSE value on variations in the time to decant for the sample. As can be observed, the RMSE value increase significantly between the baseline time to decant and times greater than 3 hours.

FIG. 9D depicts a dependence of an RMSE value on variations in the time to freeze at −80 C for the sample. As can be observed, the RMSE value increase between the baseline time to freeze and times greater than 3 hours, with a substantial increase between the baseline time to freeze and a time to freeze of 24 hours.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

Example embodiments are described above with reference to flowchart illustrations or block diagrams of methods, apparatus (systems) and computer program products. It will be understood that each block of the flowchart illustrations or block diagrams, and combinations of blocks in the flowchart illustrations or block diagrams, can be implemented by computer program product or instructions on a computer program product. These computer program instructions may be provided to a processor of a computer, or other programmable data processing apparatus to produce a machine, such that the instructions, which execute via the processor of the computer or other programmable data processing apparatus, create means for implementing the functions/acts specified in the flowchart or block diagram block or blocks.

These computer program instructions may also be stored in a computer readable medium that can direct one or more hardware processors of a computer, other programmable data processing apparatus, or other devices to function in a particular manner, such that the instructions stored in the computer readable medium form an article of manufacture including instructions that implement the function/act specified in the flowchart or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions that execute on the computer or other programmable apparatus provide processes for implementing the functions/acts specified in the flowchart or block diagram block or blocks.

Any combination of one or more computer readable medium(s) may be utilized. The computer readable medium may be a non-transitory computer readable storage medium. In the context of this document, a computer readable storage medium may be any tangible medium that can contain or store a program for use by or in connection with an instruction execution system, apparatus, or device.

Program code embodied on a computer readable medium may be transmitted using any appropriate medium, including but not limited to wireless, wireline, optical fiber cable, RF, IR, etc., or any suitable combination of the foregoing.

Computer program code for carrying out operations, for example, embodiments may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The flowchart and block diagrams in the figures illustrate examples of the architecture, functionality, and operation of possible implementations of systems, methods, and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts, or combinations of special purpose hardware and computer instructions.

It is understood that the described embodiments are not mutually exclusive, and elements, components, materials, or steps described in connection with one example embodiment may be combined with, or eliminated from, other embodiments in suitable ways to accomplish desired design objectives.

The embodiments may further be described using the following clauses:
1. A system for validating proteomics models, comprising: at least one processor; and
at least one non-transitory computer-readable medium containing instructions that, when executed by the at least one processor, cause the system to perform operations comprising: obtaining a control dataset including protein level measurements; obtaining a treatment dataset including protein level measurements; obtaining a training dataset including protein level measurements; estimating protein level noise using the control dataset and the treatment dataset; generating a validation dataset using the training dataset and the estimated protein level noise, the validation dataset including adjusted protein level measurements; generating a set of predictions by applying the validation dataset to a proteomics model trained using the training dataset; determining a validity indication using the set of predictions; and providing the validity indication.
2. The system of clause 1, wherein: obtaining the control dataset comprises assaying a first aliquot to obtain first protein level measurements; obtaining the treatment dataset comprises assaying a second aliquot to obtain second protein level measurements; and wherein: the first aliquot and the second aliquot were taken from samples having different sample characteristics; the first aliquot and the second aliquot were taken from samples handled differently; or the first aliquot and the second aliquot were subject to different assay protocols.
3. The system of clause 2, wherein: the first aliquot and the second aliquot were taken from the samples having different sample characteristics; and the sample characteristics differ in at least one of: citrate plasma or EDTA plasma composition; fed or fasted state of a patient providing the sample; serum versus plasma composition; or a presence or absence of an interference agent.
4. The system of clause 3, wherein: the sample characteristics differ in the presence of absence of the interference agent; and the interference agent includes at least one of non-steroidal anti-inflammatory drugs (NSAIDs); birth control medications; blood pressure medications; mental health medications; cholesterol medications; asthma medications; diabetes medications; thyroid medications; or anti-viral or antibacterial medications.
5. The system of any one of clauses 2 to 4, wherein: the first aliquot and the second aliquot were taken from samples handled differently; and the sample handling differs in at least one of: time between sample collection and sample freezing; temperature of sample freezing; duration of time at freezing temperature; number of freeze/thaw cycles for the sample; time to spin, the samples handled differently being plasma samples; or time to clot, the samples handled differently being serum samples.
6. The system of any one of clauses 2 to 4, wherein: the first aliquot and the second aliquot were subject to different assay protocols; and the different assay protocols differed in at least one of: reagents used; dilutions of the reagents used; steps in the assay protocols; devices used to perform the assay protocols; or a number of proteins assayed by the assay protocols.
7. The system of any one of clauses 1 to 6, wherein: the training dataset is obtained in a first context; and the treatment dataset is obtained in a second context that differs from the first context.
8. The system of any one of clauses 1 to 7, wherein: the proteomics model is trained to predict a health outcome; and a proportion of entries in the training dataset corresponding to patients experiencing the health outcome is greater than a proportion of entries in the treatment dataset corresponding to patients experiencing the health outcome.
9. The system of any one of clauses 1 to 8, wherein: estimating protein level noise using the control dataset and the treatment dataset comprises: determining pairwise differences between protein level measurements for the control dataset and corresponding protein level measurements for the treatment dataset; and characterizing, for each protein in the control dataset, a distribution of the pairwise differences.
10. The system of any one of clauses 1 to 9, wherein: generating the validation dataset using the training dataset and the estimated protein level noise comprises: generating, for a protein level measurement of a first protein in the training dataset, a sample of the estimated protein level noise for the first protein; and adding the sample to the protein level measurement to generate a corresponding adjusted protein level measurement.
11. The system of clause 10, wherein: the estimated protein level noise comprises an estimated mean and standard deviation for the first protein; and generating the sample comprises sampling a normal distribution having the estimated mean and standard deviation for the first protein.
12. The system of any one of clauses 1 to 11, wherein: determining the validity indication using the set of predictions comprises: quantifying an agreement between the set of predictions and a corresponding set of predictions generated using the proteomics model and the training dataset; or quantifying an agreement between the set of predictions and a ground truth associated with the training dataset.
13. The system of any one of clauses 1 to 12, wherein: the operations further comprises: determining a statistic of paired difference value between: a control set of predictions generated by applying the control dataset to the proteomics model; and a treatment set of predictions generated by applying the treatment dataset to the proteomics model; and determining the statistic of paired difference value satisfies an invalidity condition; and the protein level noise is estimated in response to the satisfaction of the invalidity condition.
14. A system for developing proteomics models, comprising: at least one processor; and at least one non-transitory computer-readable medium containing instructions that, when executed by the at least one processor, cause the system to perform operations comprising: obtaining a proteomics model trained to generate a prediction based on training protein level measurements generated from training aliquots obtained in a first context; generating treatment protein level measurements from treatment aliquots or samples obtained in second contexts; and determining a dependence of the proteomics model on differences between the second contexts; and updating the proteomics model based on the determined dependence.
15. The system of clause 14, wherein: updating the proteomics model based on the determined dependence comprises: identifying a protein based on variability effect of the protein; and reducing variability effect of the protein in the proteomics model.
16. The system of any one of clauses 14 to 15, wherein: the proteomics model comprises a linear or logistic regression model, a survival model, a random forest model, a support vector machine model, or a linear discriminant analysis model.
17. The system of any one of clauses 14 to 16, wherein: the training aliquots are taken from serum samples or plasma samples.
18. The system of any one of clauses 14 to 17, wherein: the second contexts have differing sample characteristics, sample handling, or assay protocols.
19. The system of any one of clauses 14 to 18, wherein: the differences in the second contexts comprise differences in at least one of a time to freeze, a number of freeze-thaw cycles, a time to spin, a fasting time, a time to ship, a freezer storage time, a time to clot, or a time to decant.
20. The system of any one of clauses 14 to 20, wherein: determining the dependence of the proteomics model on the differences between the second contexts comprises: determining protein level noise using the treatment protein level measurements; generating validation protein level measurements using the training protein level measurements and the protein level noise; and generating predictions using the validation protein level measurements.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the invention disclosed herein. It is intended that the specification and examples be considered as exemplary only. It is also intended that the sequence of steps shown in figures are only for illustrative purposes and are not intended to be limited to any particular sequence of steps. As such, those skilled in the art can appreciate that these steps can be performed in a different order while implementing the same method.

What is claimed is:

1. A system for validating proteomics models, comprising:
at least one processor; and
at least one non-transitory computer-readable medium containing instructions that, when executed by the at least one processor, cause the system to perform operations comprising:
obtaining a control dataset including protein level measurements;
obtaining a treatment dataset including protein level measurements;
obtaining a training dataset including protein level measurements;
estimating protein level noise using the control dataset and the treatment dataset;
generating a validation dataset using the training dataset and the estimated protein level noise, the validation dataset including adjusted protein level measurements;
generating a set of predictions by applying the validation dataset to a proteomics model trained using the training dataset;
determining a validity indication using the set of predictions; and
providing the validity indication.

2. The system of claim 1, wherein:
obtaining the control dataset comprises assaying a first aliquot to obtain first protein level measurements;
obtaining the treatment dataset comprises assaying a second aliquot to obtain second protein level measurements; and
wherein:
the first aliquot and the second aliquot were taken from samples having different sample characteristics;
the first aliquot and the second aliquot were taken from samples handled differently; or
the first aliquot and the second aliquot were subject to different assay protocols.

3. The system of claim 2, wherein:
the first aliquot and the second aliquot were taken from the samples having different sample characteristics; and
the sample characteristics differ in at least one of:
citrate plasma or EDTA plasma composition;
fed or fasted state of a patient providing the sample;
serum versus plasma composition; or
a presence or absence of an interference agent.

4. The system of claim 3, wherein:
the sample characteristics differ in the presence or absence of the interference agent; and
the interference agent includes at least one of.
non-steroidal anti-inflammatory drugs (NSAIDs);
birth control medications;
blood pressure medications;
mental health medications;
cholesterol medications;
asthma medications;
diabetes medications;
thyroid medications; or
anti-viral or antibacterial medications.

5. The system of claim 2, wherein:
the first aliquot and the second aliquot were taken from samples handled differently; and
the sample handling differs in at least one of:
time between sample collection and sample freezing;
temperature of sample freezing;
duration of time at freezing temperature;
number of freeze/thaw cycles for the sample;
time to spin, the samples handled differently being plasma samples; or
time to clot, the samples handled differently being serum samples.

6. The system of claim 2, wherein:
the first aliquot and the second aliquot were subject to different assay protocols; and
the different assay protocols differed in at least one of:
reagents used;
dilutions of the reagents used;
steps in the assay protocols;
devices used to perform the assay protocols; or
a number of proteins assayed by the assay protocols.

7. The system of claim 1, wherein:
the training dataset is obtained in a first context; and
the treatment dataset is obtained in a second context that differs from the first context.

8. The system of claim 1, wherein:
the proteomics model is trained to predict a health outcome; and
a proportion of entries in the training dataset corresponding to patients experiencing the health outcome is greater than a proportion of entries in the treatment dataset corresponding to patients experiencing the health outcome.

9. The system of claim 1, wherein:
estimating protein level noise using the control dataset and the treatment dataset comprises:
determining pairwise differences between protein level measurements for the control dataset and corresponding protein level measurements for the treatment dataset; and
characterizing, for each protein in the control dataset, a distribution of the pairwise differences.

10. The system of claim 1, wherein:
generating the validation dataset using the training dataset and the estimated protein level noise comprises:
generating, for a protein level measurement of a first protein in the training dataset, a sample of the estimated protein level noise for the first protein; and
adding the sample to the protein level measurement to generate a corresponding adjusted protein level measurement.

11. The system of claim 10, wherein:
the estimated protein level noise comprises an estimated mean and standard deviation for the first protein; and
generating the sample comprises sampling a normal distribution having the estimated mean and standard deviation for the first protein.

12. The system of claim 1, wherein:
determining the validity indication using the set of predictions comprises:
quantifying an agreement between the set of predictions and a corresponding set of predictions generated using the proteomics model and the training dataset; or
quantifying an agreement between the set of predictions and a ground truth associated with the training dataset.

13. The system of claim 1, wherein:
the operations further comprises:
determining a statistic of paired difference value between:
a control set of predictions generated by applying the control dataset to the proteomics model; and
a treatment set of predictions generated by applying the treatment dataset to the proteomics model; and
determining the statistic of paired difference value satisfies an invalidity condition; and
the protein level noise is estimated in response to the satisfaction of the invalidity condition.

14. A system for developing proteomics models, comprising:
at least one processor; and
at least one non-transitory computer-readable medium containing instructions that, when executed by the at least one processor, cause the system to perform operations comprising:
obtaining a proteomics model trained to generate a prediction based on training protein level measurements generated from training aliquots obtained in a first context;
generating treatment protein level measurements from treatment aliquots or samples obtained in second contexts; and
determining a dependence of the proteomics model on differences between the second contexts; and
updating the proteomics model based on the determined dependence.

15. The system of claim 14, wherein:
updating the proteomics model based on the determined dependence comprises:
identifying a protein based on variability effect of the protein; and
reducing variability effect of the protein in the proteomics model.

16. The system of claim 14, wherein:
the proteomics model comprises a linear or logistic regression model, a survival model, a random forest model, a support vector machine model, or a linear discriminant analysis model.

17. The system of claim 14, wherein:
the training aliquots are taken from serum samples or plasma samples.

18. The system of claim 14, wherein:
the second contexts have differing sample characteristics, sample handling, or assay protocols.

19. The system of claim 14, wherein:
the differences in the second contexts comprise differences in at least one of a time to freeze, a number of freeze-thaw cycles, a time to spin, a fasting time, a time to ship, a freezer storage time, a time to clot, or a time to decant.

20. The system of claim 14, wherein:
determining the dependence of the proteomics model on the differences between the second contexts comprises:
determining protein level noise using the treatment protein level measurements;
generating validation protein level measurements using the training protein level measurements and the protein level noise; and
generating predictions using the validation protein level measurements.
